(19) United States
(12) Patent Application Publication
(10) Pub. No.: US 2006/0090763 A1
(43) Pub. Date: May 4, 2006

(54) Disposable ear plug

(76) Inventor: Kwan-Ho Kim, Seoul (KR)

Correspondence Address: JORDAN AND HAMBURG LLP, 122 EAST 42ND STREET, SUITE 4000, NEW YORK, NY, 10168, US

(21) Appl. No.: 10/535,645

(22) PCT Filed: Mar. 3, 2003
(86) PCT No.: PCT/KR03/00404

§ 371 Date: May 19, 2005

(30) Foreign Application Priority Data
Jan. 9, 2003 (KR) 10-2003-0001347

(51) Int. Cl. A61F 11/00; A61F 11/06

(52) U.S. Cl. 128/864; 128/865; 128/867

(57) ABSTRACT

Disclosed is a disposable ear plug for protecting a user's ears from noise. The disposable ear plug which includes a disposable soundproofing sheet (10) including a base sheet (15) such as a crumplable paper and an artificial cell membrane containing water or oil. The disposable soundproofing sheet is irregularly crumpled at the center thereof to form a crumpled part (18) and the crumpled part is inserted in a depression of an auricle while wholly covering an ear, thereby shielding the user's ears from unpleasant noises while allowing the user to hear attenuated sounds and protecting the user from earaches caused by the insertion of the ear plug into an external auditory canal of the ear, and preventing discomfort from the sense of an alien substance embedded in the ear. Furthermore, the degree of noise attenuation is controlled by adjusting the crumpling of the soundproofing sheet.

### TECHNICAL FIELD

The present invention pertains, in general, to a disposable ear plug for protecting a user's ears from noise. More particularly, the present invention relates to a disposable ear plug which comprises a disposable soundproofing sheet including a crumplable paper or an artificial cell membrane containing water or oil. The disposable soundproofing sheet is irregularly crumpled at the center part thereof to be inserted into an auricle while wholly covering an ear, thereby shielding a user's ears from unpleasant noises while allowing the user to hear attenuated sounds and protecting the user from earaches caused by a sudden atmospheric pressure change and the insertion of the ear plug into an external auditory canal of the ear.

### BACKGROUND ART

As well known to those skilled in the art, passengers in airplanes may be bothered by various noises and feel an earache due to a sudden atmospheric pressure change. Additionally, operators working in construction and industrial fields frequently suffer from physical and mental ailments caused by exposure to loud noises.

Conventionally, ear plugs inserted into ears or earmuffs covering the ears are used to avoid unpleasant noises.

With reference to FIG. 1, there is illustrated a conventional ear plug 7 embedded in an ear of a user. When air in an external auditory canal 2 is evacuated after the conventional ear plug 7 including a cylindrical elastic material is compressed and inserted into the external auditory canal 2, the conventional ear plug 7 comes into tight contact with a wall of the external auditory canal 2 due to its resilient force, thereby stopping the external auditory canal 2 to protect the user from the unpleasant noises.

However, the conventional ear plug 7 prevents the user from hearing nearly all sounds, so the user may feel discomfort and be exposed to danger because he does not hear any sound.

For this reason, the conventional ear plug 7 includes a bore 8 positioned therethrough at the cross sectional center thereof and a thin film or a filter part 9 embedded in the bore 8, thereby allowing the user to hear attenuated sounds.

However, the user feels discomfort when the conventional ear plug 7 is embedded in the ear because of the abrasion of the wall of the external auditory canal by the conventional ear plug 7 and the sense of an alien substance embedded in the ear.

Furthermore, when the conventional ear plug 7 is inserted into the external auditory canal while it is compressed to completely close the ear, the noises travel through the bore 8 into the ear while they collide with the thin film or filter part 9, thus the noises resonate to cause the user to be temporarily deafened.

Another disadvantage of the conventional ear plug 7 is that it is discomfort to control the degree of noise attenuation using the conventional ear plug 7 according to situations or environments which the user faces.

Meanwhile, the above earmuffs are disadvantageous in view of the desirable noise prevention because they are mostly used to keep ears of the user warm and prevent the user from hearing nearly all sounds.

### DISCLOSURE OF THE INVENTION

Accordingly, the present invention has been made keeping in mind the above problems occurring in the prior art, and an object of the present invention is to provide a disposable ear plug, which protects users from unpleasant noises while allowing some necessary sounds to travel into the user's ear, and prevents the users from suffering from the noises even though nothing is inserted into external auditory canals of the users so as to close ears of the users, thereby allowing the users to feel comfort without an earache nor the sense of alien substances embedded in the ears.

It is another object of the present invention to provide a disposable ear plug, which controls the degree of noise attenuation to increase its utility.

Based on the present invention, the above objects can be accomplished by providing a disposable ear plug comprising a disposable soundproofing sheet including a base sheet such as a crumplable paper or an artificial cell membrane. The base sheet contains water and oil blended with each other by using an emulsifier, and the disposable soundproofing sheet is irregularly crumpled at the center part thereof to be inserted into a depression of an auricle while wholly covering an ear, thereby shielding a user's ear from the unpleasant noises.

### BRIEF DESCRIPTION OF THE DRAWINGS

The above and other objects, features and other advantages of the present invention will be more clearly understood from the following detailed description taken in conjunction with the accompanying drawings, in which:

FIG. 1 illustrates a conventional ear plug embedded in an ear of a user;

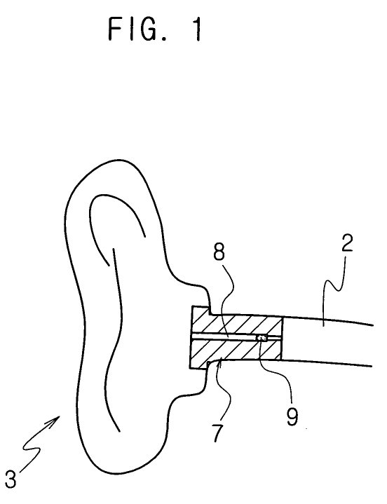

FIG. 2 is a front view of a disposable ear plug according to a first embodiment of the present invention;

FIG. 3 illustrates the disposable ear plug, worn by a user, according to the present invention;

FIGS. 4 and 5 illustrate a disposable ear plug according to a second embodiment of the present invention; and

FIGS. 6 and 7 illustrate a disposable ear plug according to a third embodiment of the present invention.

### BEST MODE FOR CARRYING OUT THE INVENTION

Reference now should be made to the drawings, in which the same reference numerals are used throughout the different drawings to designate the same or similar components.

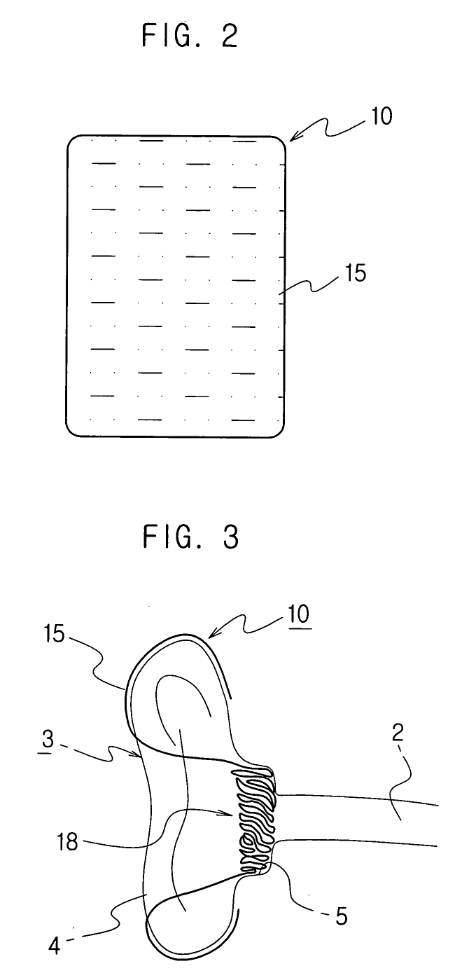

With reference to FIG. 2, there is illustrated a front view of a disposable ear plug according to a first embodiment of the present invention. The disposable ear plug includes a disposable soundproofing sheet 10 made of a base sheet 15 such as a crumplable paper or an artificial cell membrane (for example, cellulose). The base sheet 15 contains water and oil blended with each other by using an emulsifier.

At this time, only oil such as emulsion oil, or only water may be contained in the base sheet 15.

The emulsifier functions to desirably blend oil with water to uniformly disperse a mixture of oil and water in the base sheet 15, and examples of the emulsifier include sorbitan monostearate, sorbitan sequioleate, polysorbate 20, and polyoxyethylene (20) sorbitan monostearate.

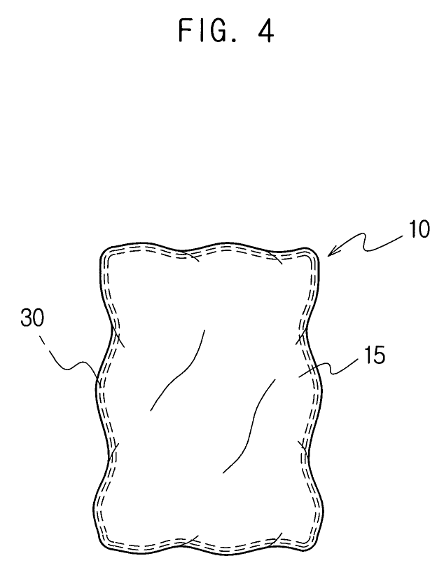

The disposable ear plug is worn by a user in such a way that the soundproofing sheet 10 covers a whole ear as shown in FIG. 3.

At this time, a girth part of the soundproofing sheet 10 is properly folded so that the soundproofing sheet 10 desirably covers the whole ear.

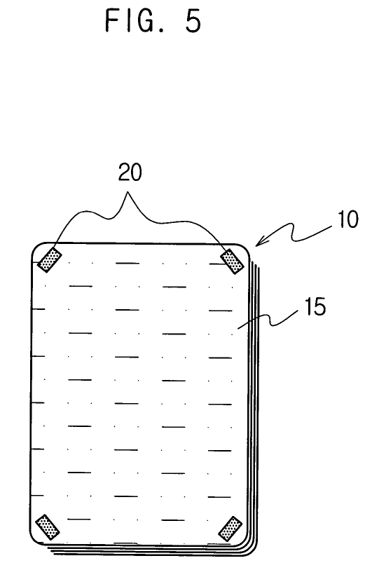

According to a second embodiment of the present invention, attachment parts 20 may be attached to corners of the soundproofing sheet 10, or an elastic band may be incorporated in the girth part of the soundproofing sheet 10 so that the soundproofing sheet 10 desirably covers the whole ear, as shown in FIGS. 4 and 5.

After the girth part of the soundproofing sheet 10 is folded in such a way that it covers an auricle of the ear, the central part of the soundproofing sheet 10 worn by the user is irregularly crumpled by the user's hands to form a crumpled part 18, and the crumpled part 18 of the soundproofing sheet 10 is then inserted into a depression of the auricle.

As described above, the ear plug according to the first embodiment of the present invention protects the user from unpleasant noises without being inserted into an external auditory canal 2 of the user, thus allowing the user to feel comfort without an earache nor the sense of alien substances embedded in the ear.

There will be given a detailed description of the procedure of protecting the user from noises using the disposable ear plug of the present invention, below.

The unpleasant noises collected by an auricle do not completely penetrate through the crumpled part 18. In other words, the crumpled part 18 functions to prevent the noises from traveling through the crumpled part 18 into the external auditory canal 2 because it is formed by irregularly crumpling the soundproofing sheet 18 to fold a piece of the soundproofing sheet 18 over and over again, and water or oil is incorporated in the crumpled part 18.

Meanwhile, the crumpled part 18 does not come into tight contact with the depression 5 of the auricle but is loosely inserted in it, so sounds travel through an interval between the depression 5 and the crumpled part 18 into the external auditory canal.

Therefore, the disposable ear plug of the present invention functions to protect the user from the unpleasant noises while allowing the user to hear attenuated sounds.

Moreover, the user can control the degree of noise attenuation by using the disposable ear plug. In detail, the degree of noise attenuation is controlled by adjusting the crumpling of the soundproofing sheet 10 when the central part of the soundproofing sheet 10 is irregularly crumpled.

In other words, when the soundproofing sheet is crumpled to a high degree, little sound penetrates through the external auditory canal of the user. On the other hand, when the soundproofing sheet is crumpled to a low degree, more sound penetrates therethrough.

Furthermore, the disposable ear plug according to the second embodiment of the present invention comprises a plurality of soundproofing sheets 10 detachably layered with each other, so the user may use the desired number of soundproofing sheets 10 as occasion demands, as shown in FIG. 4.

Meanwhile, the disposable ear plug of the present invention may be applied to protect the user from earaches caused by a sudden atmospheric pressure change, in addition to preventing discomfort due to the insertion of the disposable ear plug into the external auditory canal.

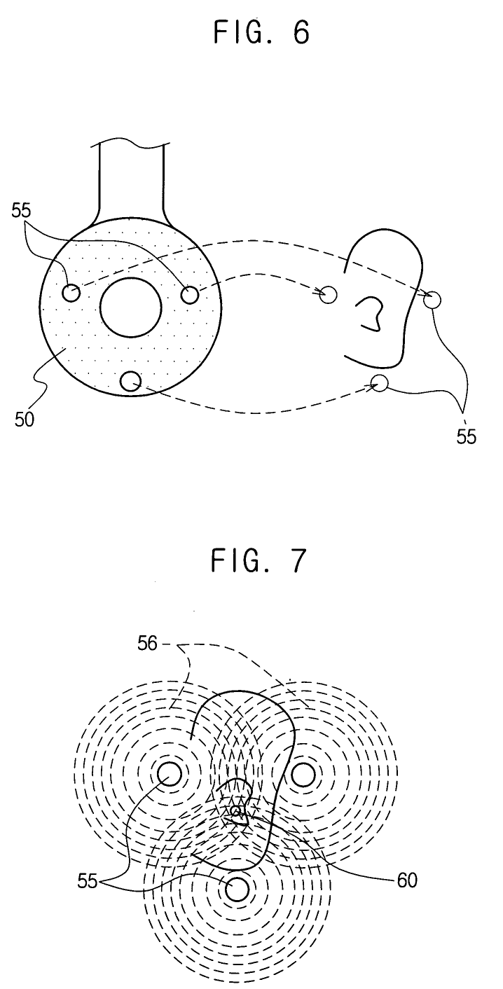

In detail, a disposable ear plug according to a third embodiment of the present invention is assembled with a traditional headset provided with a pair of adherence units 50 for covering ears of a user as shown in FIGS. 6 and 7. When the headset is worn by the user so as to allow the adherence units 50 to come into contact with the ears of the user, medical magnets 55 correspond in position to Poonji-acupoints, Yeopoong-acupoints, and Cheongung-acupoints around the ears so that eardrums of the user are located in a portion in which magnetic fields of the medical magnets 55 are overlapped with each other.

At this time, it is necessary to use the medical magnets 55 having magnetic field strength (gauss) harmless to humans, and an electromagnet capable of adjusting the magnetic field strength may be alternatively utilized.

Because effects of magnetic fields on humans depend on physical constitution, it is most preferable to use the electromagnet capable of adjusting the magnetic field strength. The magnetic field strength of the electromagnet is adjusted by a control device utilizing batteries as a power source.

Moreover, it is not necessary to locate the medical magnets 55 so as to correspond to the Poonji-acupoints, Yeopoong-acupoints, and Cheongung-acupoints around the ears, and the medical magnets may be positioned on any points so long as the eardrums 60 are located in an overlapped portion of the magnetic fields of the medical magnets.

As described above, the medical magnets 55 or the electromagnet generate magnetic fields 56.

The magnetic fields 56 are partially overlapped with each other to interfere with each other to maximize magnetic energy.

The magnetic energy functions to prevent increased atmospheric pressure from being applied to the eardrums 60.

Accordingly, when the user wears the disposable ear plug having the medical magnets 55, the sudden atmospheric pressure change does not have a bad effect on his eardrums during taking-off and landing of an airplane, and the present inventor confirmed that when wearing the disposable ear plug having the commercial medical magnets rode in an airplane, the present inventor did not keep being deafened due to a sudden atmospheric pressure change during taking-off and landing of the airplane.

### INDUSTRIAL APPLICABILITY

As described above, the present invention provides a disposable ear plug which includes a disposable soundproofing sheet made of a base sheet such as a crumplable paper and an artificial cell membrane containing water or oil. The disposable soundproofing sheet is irregularly crumpled at the center part thereof to be inserted in a depression of an auricle while wholly covering an ear, thereby shielding an user from the unpleasant noises while allowing the user to hear attenuated sounds and protecting the user from earaches caused by the insertion of the ear plug into an external auditory canal of the ear, and preventing discomfort from the sense of an alien substance embedded in the ear.

Moreover, the degree of noise attenuation is controlled by adjusting the crumpling of the soundproofing sheet 10 constituting the disposable ear plug when the central part of the soundproofing sheet 10 is irregularly crumpled, thereby utility of the disposable ear plug is desirably increased.

Furthermore, medical magnets assembled with the disposable ear plug generates magnetic fields, and the magnetic fields are partially overlapped with each other to interfere with each other to maximize magnetic energy. The magnetic energy functions to prevent increased atmospheric pressure from being applied to the eardrums 60, thereby protecting the user from earaches.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

1. A disposable ear plug comprising:
a disposable soundproofing sheet including a base sheet made of a crumplable paper or an artificial cell membrane, said base sheet containing water and oil blended with each other by using an emulsifier, said disposable soundproofing sheet being irregularly crumpled at the center part thereof to be inserted in a depression of an auricle while wholly covering an ear, thereby shielding a user from unpleasant noises.

2. The disposable ear plug as set forth in claim 1, wherein the base sheet of the disposable soundproofing sheet contains only oil.

3. The disposable ear plug as set forth in claim 1, wherein the base sheet of the disposable soundproofing sheet contains only water.

4. The disposable ear plug as set forth in claim 1, wherein the disposable soundproofing sheet includes attachment parts attached to corners thereof.

5. A disposable ear plug assembled with a headset equipped with a pair of adherence units for covering ears of a user and a plurality of medical magnets attached at regular intervals to surfaces of the adherence units so that eardrums of the user are located in a portion in which magnetic fields emitted from the medical magnets are overlapped with each other.

6. The disposable ear plug as set forth in claim 5, wherein the medical magnets attached to the adherence units correspond in position to Poonji-acupoints, Yeopoong-acupoints, and Cheongung-acupoints around the ears.
